(19) United States
(12) Patent Application Publication
(10) Pub. No.: US 2002/0133134 A1
(43) Pub. Date: Sep. 19, 2002

(54) Change-N-toss detachable sacs

(76) Inventor: Kimberly Laniece Wilbon, Fort Lewis, WA (US)

Correspondence Address: KIMBERLY WILBON, P.O. BOX2128, FORT LEONARD WOOD, MO, 65473, US

(21) Appl. No.: 09/761,223

(22) Filed: Jan. 16, 2001

(51) Int. Cl. A61F 13/15; A61F 13/20

(52) U.S. Cl. 604/385.13; 604/372; 604/377; 604/385.06

(57) ABSTRACT

A detachable sack (36) for holding absorbent items which appearing as a decorative portion of a baby's pamper when attached to the back one side. The plastic detachable sack is flexible and easy to crease (32) and is firmly held close to the pamper (8) with a clear thin plastic sheet (20) holding on each angle and side. The detachable sack includes a plastic drawstring (16) of which is positioned throughout the top hem (25) of the sack which is adapted for the sanitary retrieval and disposal of absorbent materials.

### BACKGROUND—FIELD OF INVENTION

This invention relates generally to articles for baby sanitation, specifically to a disposable sack which detaches from a pamper and is used for holding absorbent materials.

### BACKGROUND—DESCRIPTION OF PRIOR ART

The human population increases substantially each day; it's a known fact that babies are being born everyday. As the number of babies born increases, the demand for quality hygiene disposition increases as well, to prevent the spread of germs and polluting the atmosphere.

Currently, suppliers have created numerous kits and apparatus' as a sanitary precaution for baby care. To improve this problem, there have been many inventions and ideas concerned with apparatus' that allows baby supplies to be held and/or carried. For example, in U.S. Pat. No. 5,443,161 to Jonese (1995) discloses a kit containing a plurality of baby care supplies enclosed; however, these enclosures are not guaranteed to meet each consumer needs. U.S. Pat. No. 5,765,339 to Garland (1998) shows a diaper pail which is manufactured for soiled diapers that will eventually cause the consumer to be exposed to dampness and odor upon retrieval of soiled diapers. Unfortunately, the diaper pail is a large and awkwardly configured apparatus that is used primarily at the home. Thus, unless the consumer plans to use the diaper pail, he is unlikely to carry and have it available in public for use when needed. When consumers are limited to and/or have no option to disposing properly the consumer will alternatively use paper bags, newspapers, paper tissues, and other non-sanitized materials. None of these alternatives solve the problem of unplanned occurrences with pampers containing waste, because none of these items are designed to prevent contamination or hold in odors. Due to unplanned circumstances of soiled and/or waste pampers, the pamper is commonly trashed with little regard for others and the risk of polluting the atmosphere.

Accordingly, there is a need in the art for a baby sanitation article that solve these problems of (a) unavailability, (b) awkwardness and inconvenience, (c) carrying an extra apparatus, and (d) the offensiveness associated with inhaling and exhaling trash cans filled with uncovered soiled baby pampers.

Presently, these unresolved problems and deficiencies are clearly felt in the art, but nevertheless these baby supply apparatus heretofore known suffer from a number of disadvantages:

(a) The kits and apparatus' of the types in present use are not designed for immediate disposing of singular diminutive items discreetly.

(b) Their manufacture in size does not allow convenience and is not easily accessible.

(c) The kits and apparatus' in present use does not meet the consumer's needs for disposing of waste materials in an easy and convenient manner.

(d) The kit of the type presently used provides only two plastic bags that can be depleted after a couple of uses with no additional bags for other unplanned occurrences.

### OBJECTS AND ADVANTAGES

Thus, furthermore the objects and advantages of the detachable sacks described in my above patent, several objects and advantages of the present invention are:

(a) to provide a sanitize development whose production allows a more convenient and discreet way to be in possession of a disposable sack at all times as long as the consumer have pampers;

(b) to provide an easier way for consumers to properly and efficiently dispose and care for the baby(ies) without neglecting;

(c) to provide a continuation of quality consideration for hygiene;

(d) to provide a suitable and manageable method for disposing materials that contain waste which will eliminate embarrassing and uncomfortable conditions;

(e) to provide a sack designed especially in diminutive size for holding odors and terminating the spread of germs with an easier and quicker way to dispose;

(f) to provide a method that is both comfortably and conveniently to obtain;

(g) to provide a course that sufficiently meet the needs and expectations of consumers that finds it difficult to easily maintain, dispose of items properly, and continue quality care simultaneously without being exposed to leakage of absorbent items deposited into sack; and

(h) to provide a disposable non-scented and/or scented sack that is designed with a one step process with the endurance to protect against three unpleasant elements of odors, wetness, and waste.

Further objects and advantages are to provide an easy and novel technique by having a detachable sack for a more expedite and more accessible way to change and toss of absorbent materials.

### DRAWING FIGURES

In the drawings, closely related figures have the same number but different alphabetic suffixes.

FIGS. 1A to 1D shows various aspects of a sack supplied with a longitudinal drawstring that is visible on the front and back side located at the top hem of the sack.

FIG. 2 shows the front side of a pamper.

FIG. 3A shows the back side of a pamper with a detachable sack which is held with a clear plastic square sheet on one side only.

FIG. 3B shows a similar pamper with the clear plastic sheet being peeled off the detachable sack for accessibility.

FIG. 4 shows the sack after being opened, drawstring is partially pulled and creases are visible indicating that the sack has been folded.

FIGS. 5A and 5B shows a disposable sack with the drawstring being pulled on each side and than connected at median and tied.

Reference Numerals In Drawings

| | |
|---|---|
| 8 pamper | 25 top hem |
| 10 hole | 28 tie |
| 16 drawstring | 32 crease |
| 18 tear of plastic sheet | 36 sack |
| 20 clear plastic sheet | 38 pulled |

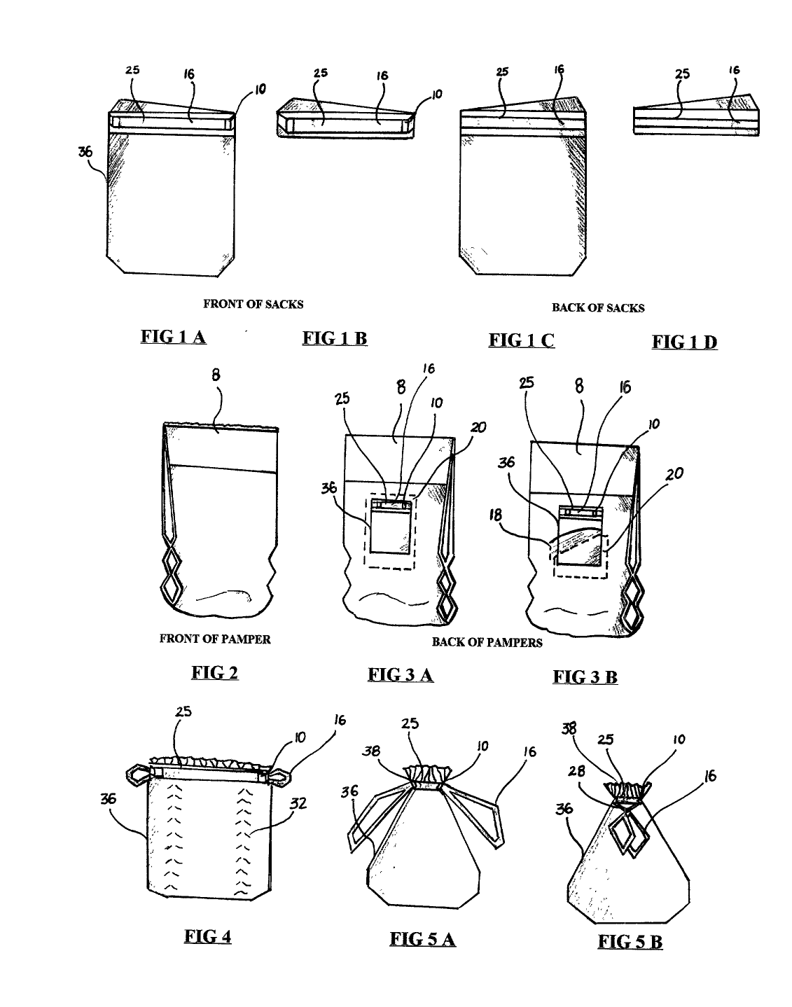

### DESCRIPTION—FIGS. 1 to 5

A typical embodiment of the sack attached to pamper of the present invention is illustrated in FIG. 3A to FIG. 3B (middle view). The sack 36 has a button hole 10 on each side of sack 36 for access to drawstring 16 which is located at the top hem 25 of sack 36 and can be repeatedly tied 28 and untied without causing damage to the top hem 25 of sack 36. A layer of clear plastic 20 is covering folded sack 36 to keep sack 36 attached to pamper 8. In the preferred embodiment, the drawstring 16 is stronger plastic made up of pol-y-eth-ylene. However, the drawstring 16 can consist of any other materials that can be tied 28 and untied without causing damage, such as rubber, leather or other fibrous materials, etc.

At the top hem 25 of the sack 36 is where the disposing item(s) are entered inside of sack 36 and the drawstring 16 is pulled 38 and tied 28. The sack's closing method, which is by drawstring 16 with a button hole 10 located at both sides of the top hem 25 of sack 36 eliminating the need for manufacturers to develop plastic ties or wire core twisters. The drawstring 16 capability also, allows consumers a more convenient manner in closing 28 and disposing of sack 36. It is an inexpensive way for manufacturers to develop diminutive sacks 36 that include a plastic tape which is position inside the top hem 25 as mentioned above.

The detachable sack 36 measurements are typically nine inches in length and nine inches in width (9 in.×9 in.) with a square shape. The outer two angles located at the top hem 25 is square to avoid shortening the drawstring 16 and weakening its function, the outer two angles located at the bottom of sack 36 are bevels shaped to give the sack 36 a distinct shape from other bags/sacks.

From the description above, a number of advantages of my detachable sacks become evident:

(a) By smaller detachable sacks being designed specifically for holding and controlling leakage of hazardous waste materials, one can simply deposit other materials inside, thus avoiding the need to pierce the link used to bind the drawstring.

(b) The detachable sacks will provide immediate odor protection which holds and eliminate contamination upon its disposition.

(c) The detachable sack does not require any kits and/or devices for availability, it's small enough to be carried inside a consumer's pocket, purse, or in hand and can be used and disposed of without causing any attention;

(d) The detachable sack is made solely to provide a safer, healthier, and proper way to dispose of items while protecting the consumer and others from wetness and waste materials.

(e) The detachable sacks are convenient for consumers that are constantly on the go and will help maintain cleanliness while properly attending to the baby(ies).

(f) The presence of a detachable sack will provide an important role in protecting the environment by reducing the risk of polluting the air.

(g) The detachable sack provides a proper way of disposing without the use of paper materials i.e., newspapers and/or paper bags that will eventually come unwrapped and does not offer the durability of holding odor and stopping the spread of germs.

(h) The detachable sack permits an easier and a more calmer approach to changing and disposing pampers and other absorbent materials.

(i) The detachable sack would be inexpensive to manufacture, low in cost to meet consumers economical needs, and provide quality service.

### OPERATION—FIGS. 1, 3, 4, 5

The manner of using the detachable sack to hold and dispose of diminutive absorbent items which has no similarities to the kits and apparatus' in present use. Namely, one first remove sack 36 from pamper 8 (not shown here but shown in FIG. 3B of my above patent). Next, open the sack 36 located at the top hem 25 on both equal sides which have button holes for access to the drawstring 16 by pulling 38 and then tie 28.

The drawstring can be tied and untied repeatedly for other deposits with the competence to continue protection.

### Summary, Ramifications, and Scope

Accordingly, the reader will see that the change-n-toss detachable sack of this invention can easily be obtainable and also be used to dispose of absorbent materials easily and conveniently. The detachable sack can be removed without causing damage to the pamper and the drawstring can be untied just as easily without damaging the sack and retied without causing exposure from the deposits inside. In addition, the change-n-toss detachable sack will engage in an important health factor that will assist in preventing the spread of germs. This detachable sack can be kept inside small areas apart from the pamper before and after use without requiring allot of storage space prior to it being used and/or trashed. Furthermore, the detachable sack has additional advantages in that

it permits holding and controlling leakage of hazardous waste;

it permits immediate protection by holding in odor and eliminating contamination;

it permits a convenient in size to be transported inside the pocket, purse, or diaper bag of a consumer and does not require being kept inside any kits and/or apparatus' which can cause attention upon retrieval and disposal of item(s);

it provides a safer, healthier, and proper way to dispose by protecting the consumers and others from the spread of germs;

it allows convenience for those that are constantly on the go and will help in maintaining cleanliness while properly attending to the baby(ies) in public places or at the home;

it provides and serves an important role in protecting the environment by reducing the risk of polluting the atmosphere;

it provides a proper way of disposing without the use of newspapers and paper bags that will eventually come unwrapped; it provides durability of holding in odors and terminating the spread of germs;

it provides an easier and a more calmer approach to changing and disposing pampers and other absorbent materials;

it provides an inexpensive way to be manufactured and is low in cost to accommodate the economical needs of consumers as well as providing quality service.

Although the description above contains many specificities, these should not be construed as limiting the space of the invention but merely providing illustrations of some of the presently preferred embodiments of this invention. For example, the detachable sack can be used as a sanitary precaution securing other absorbent items i.e., pampers, sanitary napkins, and other absorbent materials; the sack can be attached to the pamper with using other materials, i.e., plastic, velcro, adhesive, and etc.

Thus the scope of the invention should be determined by the applied claims and their legal equivalents, rather than by the examples given.

I claim:

1. A detachable sack that is attached to a folded piece of absorbent material for containing and disposing waste items, comprising:
(a) a flat body of plastic material composed of polymer,
(b) a square shape body of said plastic material of sufficient size for securing the use of diminutive waste materials,
(c) a white piece of cotton fabric of said detachable sack is folded and/or creased and placed on one side of said cotton fabric.

2. The detachable sack of claim 1 wherein said body of plastic material is folded at top hem of said body for drawstring which is positioned throughout said top hem of said body thereof, a button size aperture adjacent to and communicating with said drawstring on each side of said body.

3. The detachable sack of claim 1 wherein said body of material is attached to said folded piece of absorbent material firmly held close to one another with a clear sheet of thin plastic material, thereby holding close said square shape body of said plastic material at each angle,
(a) a detachable sack that is attached to said one side of said folded piece of absorbent material made of said cotton for having at each changing of said folded piece of absorbent material.

4. In a detachable sack of the type comprising a flat sheet of plastic material having a top hem with drawstring positioned throughout said top hem thereof and an aperture adjacent on each side to and communicating with said drawstring, the improvement wherein said folded detachable sack is reduced in size and is attached to pamper with a flat clear plastic sheet holding said folded detachable sack on each angle and side.

5. The detachable sack of claim 4 wherein said sheet of material is composed of polymer.

6. The detachable sack of claim 4 wherein said sheet is a sufficient size for containing used diminutive absorbent materials.

7. The detachable sack of claim 6 where in said size is formed into and along the diminutive shape for containing of said absorbent materials.

8. The sack of claim 4 wherein said drawstring has a longitudinal position which run through said top hem of said sack thereof and stretching said drawstring to connect at the medium of said sack and tied.

9. The attachment to pamper of claim 4 wherein said clear plastic sheet which is covering said detachable sack on the back one side of said pamper, by gripping the edge of said clear plastic sheet and pulling to the opposite edge to retrieve said detachable sack.
